SEQUENCE LISTING (1 sequence)

SEQ ID NO: 1 - DNA, 19 bp, Artificial
  synthetic
acgggtcaaattggccaac

(19) United States
(12) Patent Application Publication
(10) Pub. No.: US 2011/0020806 A1
(43) Pub. Date: Jan. 27, 2011

(54) Rapid DNA Sequencing by Peroxidative Reaction

(75) Inventors: Nader Pourmand, Santa Cruz, CA (US); Larissa Munishkina, Santa Cruz, CA (US); Miloslav Karhanek, Santa Cruz, CA (US)

Correspondence Address: PETERS VERNY , L.L.P., 425 SHERMAN AVENUE, SUITE 230, PALO ALTO, CA, 94306, US

(73) Assignee: THE REGENTS OF THE UNIVERSITY OF CALIFORNIA, Oakland, CA (US)

(21) Appl. No.: 12/750,104

(22) Filed: Mar. 30, 2010

Related U.S. Application Data
(60) Provisional application No. 61/165,061, filed on Mar. 31, 2009.

(51) Int. Cl. C12Q 1/68

(52) U.S. Cl. 435/6

(57) ABSTRACT

Disclosed is a method of polynucleic acid (e.g., DNA) sequencing which is based on the generation of pyrophosphate (PPi) that occurs when a complementary base is incorporated into a growing DNA strand being synthesized on a template. The method utilizes a cascade of enzymatic reactions catalyzed by hypoxanthine-phosphoribosyl transferase, xanthine oxidase, and peroxidase in addition to DNA polymerase and apyrase. The last chemical step in the cascade of reactions is the oxidation of a material such as an electrode or luminol by hydrogen peroxide. This generates a detectable electrical or optical signal. This method is independent of luciferase, does not require dATP analogue, and is intended to improve precision and sensitivity of DNA sequencing, and to lessen the unsynchronized polymerization.

### CROSS-REFERENCE TO RELATED APPLICATIONS

This application claims priority from U.S. Provisional Patent Application No. 61/165,061, filed Mar. 31, 2009, which is hereby incorporated by reference in its entirety

### STATEMENT OF GOVERNMENTAL SUPPORT

This invention was made with U.S. Government support under National Institutes of Health Grant P01-HG000205 and National Science Foundation Grant DBI 08300141. The U.S. Government has certain rights in this invention.

### REFERENCE TO SEQUENCE LISTING, COMPUTER PROGRAM, OR COMPACT DISK

None

### BACKGROUND OF THE INVENTION

1. Field of the Invention

The present invention relates to the field of chemical reactions, particularly those used in nucleic acid sequencing, more particularly to determining the sequence of e.g., DNA by generation of light in a series of reactions resulting in chemiluminescence or by an electrochemical reaction.

2. Related Art

Presented below is background information on certain aspects of the present invention as they may relate to technical features referred to in the detailed description, but not necessarily described in detail. That is, certain components of the present invention may be described in greater detail in the materials discussed below. The discussion below should not be construed as an admission as to the relevance of the information to the claimed invention or the prior art effect of the material described.

Reliable and rapid DNA sequencing is the demand of our modern society. It is crucial for the development of biological sciences, medical applications, and biotechnological innovations. During last decade, several high-throughput DNA sequencing methods have been developed and commercialized. The major DNA sequencing methods called the next-generation methods are Illumina sequencing (SOLEX), SOLID (ABI), and Pyrosequencing (Biotage/Roche). All three methods are distinct from Sanger's sequencing and are based on different approaches to sequence DNA. The Illumina method utilizes the sequential incorporation of fluorescently label nucleotides that are protected on the 3′ end and cannot be extended by DNA polymerase, only after luminescence is detected, the protected group is released by laser scission, and the next nucleotide can be incorporated into the growing DNA chain. (See U.S. Pat. No. 6,355,431) The drawbacks of Illumina approach are the cost of double-labeled fluorescent nucleotides and their effects on DNA polymerase processivity and accuracy. SOLID method (See WO/2006/084132) involves sequential DNA ligation of fluorescently labeled oligonucleotides and removal of the labels by exonuclease after the detection of fluorescence that allowing ligation of the next oligonucleotides. The drawbacks of SOLID are low processivity and costly fluorescently labeled oligonucleotides.

Pyrosequencing is based on the sequential detection of pyrophosphate released during DNA polymerization. Pyrophosphate release is coupled to light emission through a cascade of enzymatic reactions catalyzed by APS tranferase and luciferase. In comparison to SOLID and Illumina methods, Pyrosequencing has higher processivity, but lacks accuracy in homopolymeric regions and requires the tandem addition of nucleotides (dNTPs are added one at a time, not as a mixture). Due to the development of emulsion PCR and bridge PCR, clusters of unique DNA molecules can be generated and deposited on the glass slides at very high density allowing parallel reading of millions of DNA clusters at a time. Parallel reading of millions of DNA clusters results in high throughput of these next generation methods that significantly reduces the cost of sequencing per base and makes them leaders in DNA sequencing technology today. However, the cheap sequencing is not good enough for de novo sequencing and may have restriction in the resequencing applications due to the bias of assembly of genomes from short DNA readouts. It is already feasible to conclude that the next generation methods produce tremendous amount of raw data that are not yet assembled and require complicated hardware setup such as clusters as well as the development of complex bioinformatic software. Even if these requirements are fulfilled the problem of bias assembly will persist.

The third-generation DNA sequencing methods are emerging on the market. Their approaches are based on the single-molecule detection and the extension of DNA reading length. True single-molecule sequencing (tSMS) of Helicos mostly focus on single molecule detection, where as FRET-based approach of VisiGen Biotechnologies, single-molecule real-time sequencing of Pacific Biosciences, nanopore sequencing (various approaches and companies), and transmission electron microscopy (TEM) of ZS Genetics provide in addition to single-molecule sequencing a long readout of DNA. Nonetheless, the third generation methods are still under development and would require significant technological improvements before they will be fully used. The exception is tSMS of Helicos that is already on the market. However, Helicos method is not significantly different from Illumina method, and as expected the cost of the sequencing as well as the reading length are the same as that of the second-generation DNA sequencing methods.

The goal of the sequencing technologies is to provide cheap (<$10,000/109 bases), accurate (10⁻⁵ mistake/base), fast (10⁹-10¹¹ bases/day), and representative (>98% coverage). DNA sequencing may be achieved by using one technology or it may be achieved by a combination of the methods. The drawbacks and limitations of pyrosequencing are mostly related to the reading of homopolymeric regions, asynchronous DNA polymerization, and the usage of dATP analogue dATP-alpha-S. The usage of thio-dATP as the substrate for DNA polymerase causes the decrease in the rate of DNA polymerization and sequentially increases asynchronous DNA polymerization especially in the homopolymeric poly(dT) regions. The substitution of the luciferase cascade of pyrosequencing to a different set of enzymatic reactions that is only sensitive to pyrophosphate and not sensitive to dATP or any other component of DNA polymerization reaction will be advantageous because it will eliminate or greatly diminish the limitations of pyrosequencing.

### SPECIFIC PATENTS AND PUBLICATIONS

Ronaghi, M. “Pyrosequencing sheds light on DNA sequencing,” Genome Res. Ian; 11 (1):3-11 (2001) discloses pyrosequencing detection methods using a bioluminometric detection is a three step reporter technique.

U.S. Pat. No. 7,141,370 to Hassibi, et al., issued Nov. 28, 2006, entitled “Bioluminescence regenerative cycle (BRC) for nucleic acid quantification,” discloses methods of quantifying nucleic acids using a bioluminescence regenerative cycle (BRC). In BRC, steady state levels of bioluminescence result from processes that produce pyrophosphate. Pyrophosphate reacts with APS in the presence of ATP sulfurylase to produce ATP. The ATP reacts with luciferin in a luciferase-catalyzed reaction, producing light and regenerating pyrophosphate.

U.S. Pat. No. 6,210,891 to Nyren, et al., issued Apr. 3, 2001, entitled “Method of sequencing DNA,” discloses a method of DNA sequencing in which, in place of deoxy- or dideoxy adenosine triphosphate (ATP), a dATP or ddATP analogue is used which is capable of acting as a substrate for a polymerase but incapable of acting as a substrate for a said PPi-detection enzyme and wherein release of PPi is indicative of incorporation of deoxynucleotide or dideoxynucleotide and the identification of a base complementary thereto.

US 2006/0105373 A1 by Pourmand et al., entitled “Charge perturbation detection system for DNA and other molecules,” published May 18, 2006, discloses a method which may be used for sequencing in which electric charge perturbations of the local environment during enzyme-catalyzed reactions are sensed by an electrode system.

US 2004/0248227, Jansson, et al., published Dec. 9, 2004, entitled “Enzymatic determination of inorganic pyrophosphate,” discloses a method for determining inorganic pyrophosphate in a sample, which method comprises contacting the sample with an aqueous reagent comprising xanthosine 5′-monophosphate (XMP) or preferably inosine 5′-monophosphate (IM), xanthosine phosphoribosyltransferase or preferably hypoxanthine phosphoribosyltransferase, xanthine oxidase, a divalent cation which is preferably Mg²⁺, and a buffering agent which is preferably tris(hydroxymethyl)aminomethane (Tris); and determining production of hydrogen peroxide as a measure or inorganic pyrophosphate in the sample.

### BRIEF SUMMARY OF THE INVENTION

The following brief summary is not intended to include all features and aspects of the present invention, nor does it imply that the invention must include all features and aspects discussed in this summary.

The present invention utilizes an oxidative reaction, such as chemiluminescence, in which light is detected in a series of reactions beginning with the generation of an inorganic phosphate (PPi), also known as pyrophosphate, P₂O₇⁴⁻. As is known, PPi results through the correct incorporation of a nucleotide into a growing strand. According to the present invention, the PPi is enzymatically converted into a mixture that contains an oxidative compound such as hydrogen peroxide (H₂O₂). Sufficient oxidative compound is created such that when is reacted with a suitable substrate or a chemiluminescent compound (such as luminol), it generates a detectable signal, which is then detected to provide sequence information. If an incorrect (noncomplementary) nucleotide is added to the reaction mixture, no signal (e.g., light) is emitted. Thus a template strand may be sequenced by adding all four possible bases to the reaction mixture, preferably in a predetermined order; only the correct base (pairing with the template strand) will be incorporated to emit the signal (light). As is well known in the art, in DNA, A will only pair with T; G will only pair with C. In RNA, the same rules apply, except that U is used in place of T.

In general terms, the present method employs (1) the catalyzed conversion of PPi (such as split off from the triphosphate group on a nucleotide which is added to the 3′ hydroxyl of a growing polynucleotide chain) to form an oxidation substrate (where the phosphate group is preferably transferred to another molecule); (2) the oxidation of this oxidation substrate with a concomitant production of hydrogen peroxide; and (3) the use of hydrogen peroxide to oxidize a chemiluminescent compound or otherwise participate in a redox reaction. In step (1), a phosphotransferase is used to form an oxidatable compound. That is, is used to react with the PPi and a molecule such as IMP. IMP has the structure

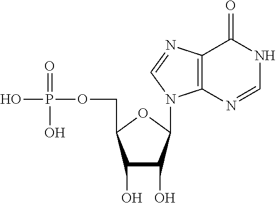

The IMP is converted to an enzymatically oxidatable hypoxanthine, which has the structure

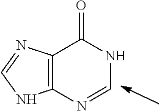

So, in step (1), an inorganic phosphate is added to a ribose moiety to result in a sugar triphosphate, phosphoribosyl pyrophosphate. The hypoxanthine illustrated above may then be further oxidized (preferably by xanthine oxidase) in the presence of oxygen and water, at the position indicated by the arrow, forming xanthine and hydrogen peroxide (H₂O₂), which is used in a chemiluminescence reaction. Oxidatable compounds can serve as substrate for, and be oxidized by, oxidases which generate hydrogen peroxide, e.g. NADH oxidase and NAD.

In one alternative embodiment, the hydrogen peroxide may be used in an oxidation reaction which is an electrochemical reaction which affects an electronic sensor. As an example, a material such as a metal electrode is oxidized along with reduction of the hydrogen peroxide. The metal electrode is part of a sensitive detecting circuit whereby a change in oxidation state is detected. The hydrogen peroxide may be sensed in a variety of ways. Hydrogen peroxide decomposes to produce O₂ and 2H⁺ which may be used to effect a current, charge or impedance on an electrode in the vicinity of the reaction.

### BRIEF DESCRIPTION OF THE DRAWINGS

FIG. 1 is a sketch showing the overall use of the present light generating reactions to sequence a polynucleotide such as DNA.

FIGS. 2A and B is a representation of preferred reactions, beginning with PPi and ending with the emission of light (FIG. 2A) and removal of a proton from a solution (FIG. 2B).

FIG. 3 is a representation of the reaction of luminol (having the illustrated structure) to emit a photon of light (hv).

FIGS. 4A and B are (4A) plots of an XOD activity assay, absorbance of HX (solid line) and product uric acid (dashed line), and (4B) kinetics of XOD measured at 290 nm.

FIGS. 5A and B are absorbance spectra and kinetics of HPRT in the presence of XOD; FIG. 5A shows absence of XOD and HPRT (black circles), XOD, HPRT and IMP (white triangles), after reaction upon addition of PPi; FIG. 5B shows kinetics of HPRT in the presence of XOD.

FIG. 6 is a graph showing chemiluminescence versus POD or POD+H₂O₂.

FIGS. 7A and B shows kinetic analysis of xanthine oxidase. (7A) Absorption spectra of hypoxanthine in the presence (middle line) and absence of xanthine oxidase (bottom line). The top line corresponds to absorption spectrum of xanthine oxidase. (7B) Kinetics of xanthine oxidation in the presence (top line) and absence of xanthine oxidase (bottom line) measured as time-dependant changes in absorbance at 270 nm.

FIGS. 8A and B are duplicate graphs showing data from pyrosequencing reactions plotting current versus time to illustrate the sensitivity of pyrosequencing to inorganic pyrophosphate. Luminescence was measured on the custom-setup luminometer made of a photodiode and Chem-Clamp amplifier. The sensitivity of the instrument and pyrosequencing reaction containing enzymes and substrates was less than 0.5 pmol of pyrophosphate (8A).

FIGS. 9A and B shows pyrophosphate detection on PSQ-96. Chemiluminescence of pyrosequencing reaction was measured in the presence of 0.5 pmol (9A) and 5 pmol of pyrophosphate (9B).

FIGS. 10A and B shows a series of current versus time peaks in an analysis of sensitivity of peroxysequencing reactions to hypoxanthine. Chemiluminescence of peroxysequencing reactions was measured on the custom-setup luminometer. Chemiluminescence was triggered by addition of 5 (A) and 0.5 pmol hypoxanthine (10B).

FIGS. 11A and B shows current versus time peaks in an analysis of sensitivity of peroxysequencing reactions to hydrogen peroxide. Chemiluminescence emission was initiated by addition of 3 (11A) and 0.3 pmol hydrogen peroxide (11B).

FIGS. 12A, B and C shows current versus time peaks in a comparison of kinetics of chemiluminescent reactions of pyrosequencing and peroxysequencing. Kinetic measurements of the oxidation of luciferol by luciferase in the presence of 3 (12A) and 0.3 pmol of ATP (12B). Fast kinetics of luminol oxidation by HRP in the presence of 0.3 pmol of hydrogen peroxide (12C).

FIGS. 13A and B shows current versus time peaks in a comparison of kinetics of pyrophosphate detection by peroxysequencing and pyrosequencing reactions. FIG. 13A: Fast kinetics of peroxysequencing reactions initialized by addition of 100 pmol of inorganic pyrophosphate (PPi). The reaction is completed in less than 5 sec interval. FIG. 13B: Kinetics of pyrosequencing reactions triggered by addition of 100 pmol of PPi. The reaction is completed in more than 30 sec interval. The low signal of peroxysequencing reaction was partially due to the presence of chloride ions in the HPRT preparation that inhibited the luminescence and to substrate inhibition.

### DETAILED DESCRIPTION OF THE PREFERRED EMBODIMENT

### Overview

The present methods utilize a DNA sequencing chemistry based on pyrophosphate detection using a new set of enzymatic reactions that may sense pyrophosphate at picomolar concentration in the real time. The present technique is an improvement on the well-known pyrosequencing technique in utilizing chemiluminescent detection assay for pyrophosphate released during DNA polymerization reaction. Pyrosequencing utilizes PPi to create ATP, which generates light when utilized in a luciferase-luciferin reaction.

The reaction catalyzed by firefly luciferase takes place in two steps:

luciferin+ATP→luciferyl adenylate+Ppi

luciferyl adenylate+O₂→oxyluciferin+AMP+light.

The prior art methods of pyrosequencing have several limitations and drawbacks such as the requirement for the substitution of dATP with the thiol-containing analogue dATP-α-S. The substitution is necessary because dATP independently of DNA polymerization activates the luciferase, which results in erroneous emission of light and sequencing errors. Furthermore, the analogue dATP-α-S interferes with DNA polymerase processivity and apyrase activity and leads to unsynchronized polymerization that affects the reading length and precision of DNA sequencing.

One objective of the present method is to overcome the drawbacks and limitations of pyrosequencing by using a different cascade of enzymatic reactions.

This method does not use luciferase and APS transferase, but utilizes a cascade of enzymatic reactions catalyzed by hypoxanthine-phosphoribosyl transferase, xanthine oxidase, and peroxidase in addition to DNA polymerase and apyrase.

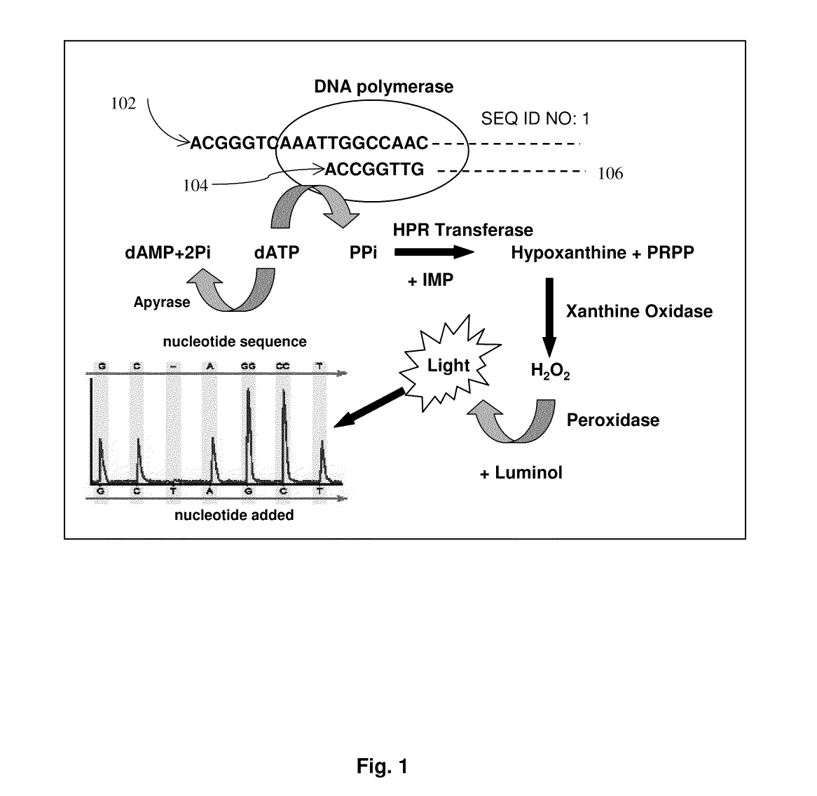

As shown in FIG. 1, the released pyrophosphate from polynucleic acid growth is recognized as a substrate by a hypoxanthine-phospho-ribosyl transferase that converts inosine-monophosphate (IMP) and pyrophosphate (PPi) into hypoxanthine and phosphoribosylpyrophosphate (PRPP). The formed hypoxanthine is oxidized by xanthine oxidase (XOD) to uric acid with the production of hydrogen peroxide. Hydrogen peroxide is a substrate of peroxidase (POD) that catalyzes the chemiluminescent reaction of oxidation of luminol to 3-aminophthalate. The emitted light can be detected by photomultipliers or arrays of diodes or photocapacitors of charge-coupled device (CCD camera). This new method of DNA sequencing is named peroxysequencing.

In addition, further peroxide may be generated because the xanthine product of the xanthine oxidase reaction shown in FIG. 1 converts in the presence of water and oxygen to urate. Urate may be optionally further oxidized by uricase to produce allantoin, CO₂ and H₂O₂. In this scheme, H₂O₂ is made in three different reactions: of hypoxanthine to xanthine; result of xanthine to urate; and result of urate to allantoin. Uricase is also important in the removal of urate, which also improves luminescence. Various enhancers for increasing the generation of activated (light emitting) luminol from luminol may be used, as well as luminol analogs (such as Sigma's CPS-2 Chemiluminescent Peroxidase Substrate-2).

Electrochemical reactions involving hydrogen peroxide may be used in place of chemiluminescence. For example, these may rely on the redox activity of hydrogen peroxide, where H+ ions are removed from an acidified solution. For example, using potassium iodide (which will generate iodine that can also be detected) the reaction is 2H⁺+H₂O₂+2I⁻→I₂+2H₂O. As another example, H₂O₂ will decompose to form water and oxygen in a redox reaction. The half reaction H₂O₂→O₂+2H⁺+2e⁻ may be sensed directly through a change in the charge, impedance or current through an electrode positioned in the reaction mixture close to the growing DNA strand. Further description of an appropriate electrical circuit may be found in Pourmand et al., “Direct electrical detection of DNA synthesis,” Proc. Nat. Acad. Sci., 103(17): 6466-6470 (2006).

### DEFINITIONS

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

The term “oxidatable compound” means a compound that can participate in a chemical reaction whereby hydrogen peroxide is formed. This is preferably an enzymatically oxidatable compound, acted upon by an oxidase (e.g. xanthine oxidase). A number of oxidases are known to catalyze reactions in which an oxygen group is reduced to hydrogen peroxide.

The term “phosphotransferase” means an enzyme in a category of enzymes (EC number 2.7) which catalyze phosphorylation reactions, where a phosphate group is removed from one molecule, here PPi, and transferred to another molecule.

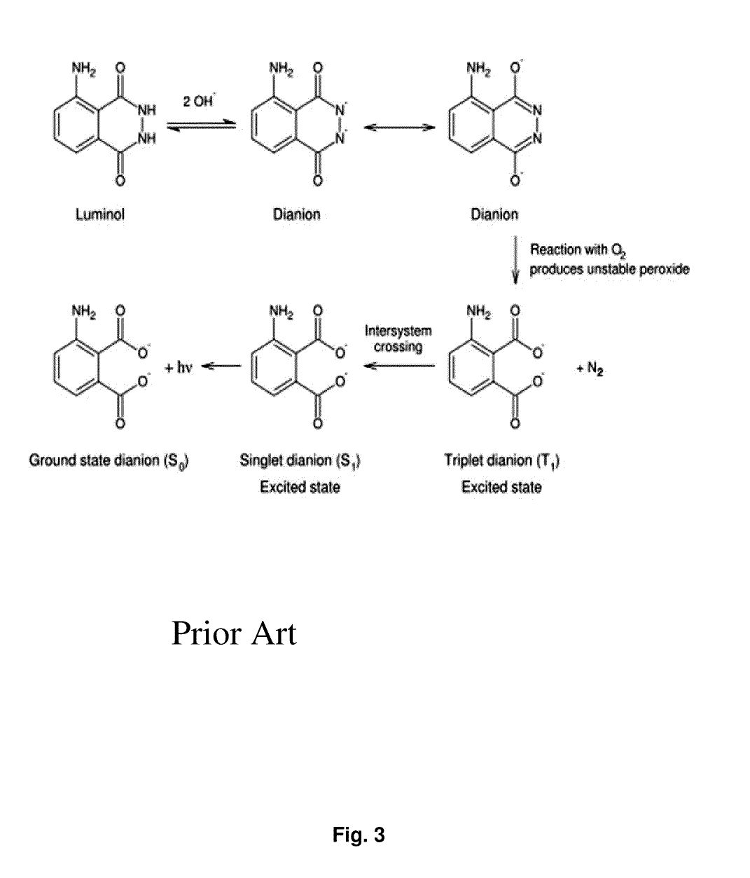

The term “luminol” is used herein to refer to chemiluminescent phthalhydrazides including luminol, or 5-amino-2,3-dihydro-1,4-phthalazinedione, which has a chemical formula as shown in FIG. 3, including a double ring structure with a melting point of about 320° C. Luminol is commercially available from several suppliers and is well characterized. The term luminol compound may also be used to include “luminol analogs” which are also chemiluminescent, such as those wherein the position of the amino group is shifted (e.g., isoluminol, the amino group being at the 6 position), or is replaced by other substituents, as well as annelated derivatives and those with substitution in the non-heterocyclic ring. Some luminol analogs produce light more efficiently than does luminol itself, while others have lower efficiency. (As used herein, the term “luminol analog” encompasses such related species.) Luminol analogs include other chemiluminescent phthalazine derivatives, e.g., as described in U.S. Pat. No. 4,011,219 to Nishii, et al., issued Mar. 8, 1977, entitled “Phthalazine derivatives and salts thereof.”

Generally, luminol produces light in an oxidizing reaction, wherein the luminol combines with oxygen or another oxidizer to produce a reaction product and photons at a wavelength of about 425-450 nanometers (nm). The precise reaction formula and the quantum efficiency of light production, i.e., the ratio of luminescing molecules to total molecules of the luminescent species, depend upon the medium in which the luminol resides, temperature and other reaction conditions. Typical oxidizers used in conjunction with luminol include oxygen, hydrogen peroxide, hypochlorite, iodine and permanganate. This reaction does not require any enzymatic or other catalysis, but is here preferably catalyzed by peroxidase acting upon peroxide and the luminol.

The term “xanthine oxidase” means an enzyme which catalyzes hypoxanthine to form uric acid and hydrogen peroxide. It is EC 1.17.3.2, CAS Registry 9002-17-9. It is available from bovine and rat sources, as well as commercially. Various forms of xanthine oxidase act on related substrates, e.g., 1,3-dimethylxanthine. If a different phosphoribosyltransferase is used, one may employ a modified form of xanthine oxidase to catalyze the next reaction in the present method, so long as hydrogen peroxide is formed.

The term “peroxidase” means an enzyme which catalyzes the breakdown of peroxide and concomitant oxidation of a donor molecule. It is EC 1.11.1.7, CAS registry 9003-99-0. Different peroxidases may be used in the present methods, including horseradish peroxidase. A presently preferred peroxidase may be obtained from Arthromyces. See, Akimoto et al., “Luminol chemiluminescence reaction catalyzed by a microbial peroxidase,” Anal. Biochem., 189(2):182-185 (1990) for a full description of this enzyme. Peroxidase is used in the present method for catalyzing the oxidation of luminol.

The term “hypoxanthine phosphoribosyltransferase” or HPRT, means an enzyme which catalyzes the addition of an inorganic phosphate to form hypoxanthine, using as a substrate inosine-monophosphate. It is EC:2.4.2.8, CAS registry No. 9016-12.0. It is also known in humans as hypoxanthine-guanine phosphoribosyltransferase (HGPRT). It may be obtained from humans, Plasmodium falciparum, E. coli, and other sources. It may be produced by recombinant DNA methods. Cloning of the HGPRT from T. cruzi is described in Mol Biochem Parasitol., 1994 June; 65(2):233-45, “Molecular characterization and overexpression of the hypoxanthine-guanine phosphoribosyltransferase gene from Trypanosoma cruzi.”

The term “uricase” refers to a urate oxidase, which catalyzes the reaction of urate+O₂+H₂O to allantoin+H₂O₂ and CO₂. It is EC 1.7.3.3, CAS Registry 9002-12-4.

Advantages of the Present Method of Chemiluminescent Sequencing with Peroxide Based Oxidation (Peroxysequencing)

Like pyrosequencing, peroxysequencing is a real-time DNA sequencing method and allows the quantitative detection of polymorphic DNA chains. Unlike pyrosequencing, which employs a dATP-sensitive luciferase reaction, peroxysequencing allows the use of dATP instead of its substitute dATP-α-S. This eliminates interference of dATP-α-S on DNA polymerase and apyrase, leads to the efficient dATP incorporation during DNA polymerization, and decreases asynchronization. The ultimate results are longer and accurate DNA readouts.

Further, the present peroxide-based methods do not require that the DNA to be sequenced be labeled, or the nucleotides being incorporated be labeled. The nucleotides may be in native form. The present methods do not require luciferase, or other enzymes that may react on ATP.

The present methods do not use APS transferase, which is used in pyrosequencing to convert converts PPi to ATP in the presence of APS. The present methods utilize a cascade of enzymatic reactions catalyzed by hypoxanthine-phosphoribosyl transferase, xanthine oxidase, and peroxidase in addition to DNA polymerase and apyrase. Because the last step in the cascade of reactions is the oxidation of luminol by hydrogen peroxide the method is termed peroxysequencing. Peroxysequencing is independent of luciferase, does not require dATP analogue, and can extend the processivity of DNA polymerase, improve precision and sensitivity of DNA sequencing, and lessen the unsynchronized polymerization. All these features make peroxysequencing superior to pyrosequencing.

### Reactions and Detection

During DNA polymerization, inorganic pyrophosphate is released if the nucleotide is complementary to the template (DNA or RNA chain) and becomes incorporated into the growing DNA chain according to the following equation (1):

DNAₙ+dNTP→DNAₙ₊₁+PPi (1);

The released pyrophosphate is detected by a series of enzymatic reactions that culminates in the production of light according to the scheme in FIG. 1.

Pyrophosphate and inosine-monophosphate (IMP) are converted into hypoxanthine and phosphoribosylpyrophosphate (PRPP) by hypoxanthine phosphoribosyltransferase according to the following equation (2):

PPi+IMP→PRPP+Hypoxanthine (2);

The released hypoxanthine is oxidized by xanthine oxidase to produce hydrogen peroxide (H₂O₂) and urate according to the following equations (3):

Hypoxanthine+H₂O+O₂→Xanthine+H₂O₂

Xanthine+H₂O+O₂→Urate+H₂O₂ (3);

Urate is further oxidized by uricase with release of hydrogen peroxide and allantoin according to the following equation (4):

Urate+2H₂O+O₂→Allantoin+CO₂+H₂O₂ (4);

Luminol in the presence of hydrogen peroxide and peroxidase is readily oxidized and forms 3-aminophthalic acid, a compound that emits light according to the following equation (5):

Luminol+H₂O₂→3-aminophthalate+N₂+light (5);

The light is detected by a CCD camera, analyzed by computer, and presented as a peak in a chromogram (or luminogram). The height of each peak is proportional to the amount of generated light as well as the number of nucleotides incorporated.

Unincorporated dNTPs are degraded by apyrase or washed away. After the reset of the reaction (completion of enzymatic steps), solution containing the next dNTP is added and the cycle repeats. dNTPs are added one at a time. The sequential addition of dNTPs results in the generation of the signal peaks that correspond to the sequence of the newly polymerized DNA chain.

Thus the present method may be carried out in a device that contains a reaction area for holding one or more template strands of DNA to be sequenced, and reagents for the incorporation of a nucleotide into a growing strand. Thus in FIG. 1, incorporation of “A” 104 took place on a template strand 102 because, A—and only A—is complementary to the T shown opposite on the template strand 102. This reaction, which generates a molecule of PPi for every molecule of nucleotide incorporated, took place because reactants and conditions to allow “sequencing by synthesis” are provided, as are known in the art. The template contains additional sequence information, e.g., the next T. The growing strand 106, will be extended in the next sequencing round, when a complementary A is incorporated. Already paired bases are shown as dashes. The circle labeled “DNA polymerase” is part of the reaction mixture and is bound to the two strands to cause the nucleotide incorporations, sequentially in a 5′ to 3′ direction, reading the parental strand 102 in a 3′ to 5′ direction.

Also as shown in FIG. 1, the generation of light resulting from the PPi is correlated to a DNA sequence as a light peak. Existing pyrosequencing equipment may be used for this purpose. As shown, an increase in light indicates the incorporation of a complementary base. In the case of repeating units, a larger peak is observed, e.g., the sequence AGGCC would yield a peak size x for A, and 2× for GG and CC.

### Examples

Enzymes XOD (xanthine oxidase) and POD (peroxidase) have been purchased from Sigma-Aldrich and tested for their maximum activity and stability at various pH, temperature, and ionic conditions. Enzyme HPRT (hypoxanthine phosphoribosyltransferase) was not commercially available and has been purified either from E. coli as the overexpressed recombinant human HPRT or from yeast S. cerevicias as the endogenous protein. All preparations of HPRT are active, however because the yield from E. coli is higher, we have used human recombinant HPRT in the majority of our assays.

Four main types of assays may be used to test activity of enzymes and determine their computability in an enzymatic cascade. The first type of assay involves testing the products of each reaction by HPLC. The majority of substrates and products including IMP, hypoxanthine, xanthine, urate, allantoin, luminol, and 3-aminophthalate have absorption in UV-vis region and can be separated on reverse phase HPLC (RP-HPLC). The usage of RP-HPLC has been important to demonstrate that the expected products are generated during reaction, to check the side-reaction products, and to ensure the purity of substrates. For instance, the application of RP-HPLC has been critical in the finding the reason of low catalytic activity of HPRT. The low catalytic activity has been due to the contamination of HPRT extracts with alkaline phosphatase that dephosphorylate IMP, the substrate of HPRT. The further purification of HPRT on GMP-agarose affinity column has eliminated the contamination with alkaline phosphatase. The second type of assay is kinetic assays of quantitative measurements of catalytic activities of enzymes. Time-dependent measurements have been done on UV-vis Shimatsu spectrophotometer. These kinetic assays are necessary for specific activity calculation and for investigation of the effects of pH, ionic strength, and potential inhibitors on rates of catalysis.

The third type of assay involves photometer measurements using a custom setup (described in detail below). A custom photometer setup is superior to pyro sequencing instrument PSQ-96 in photon detection sensitivity (−50 times). The photometer measurements are necessary for detection and recording of light generated during the chemiluminescent reaction of luminol oxidation as well as the cascade of enzymatic reactions coupled to luminol oxidation. The fourth type of experiment, done on PSQ-96, compares the sensitivity and compatibility of pyrosequencing and peroxysequencing chemiluminescent detection of pyrophosphate.

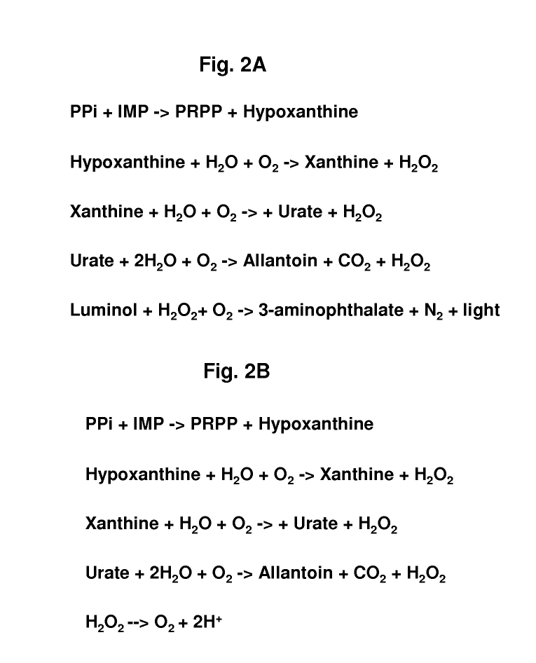

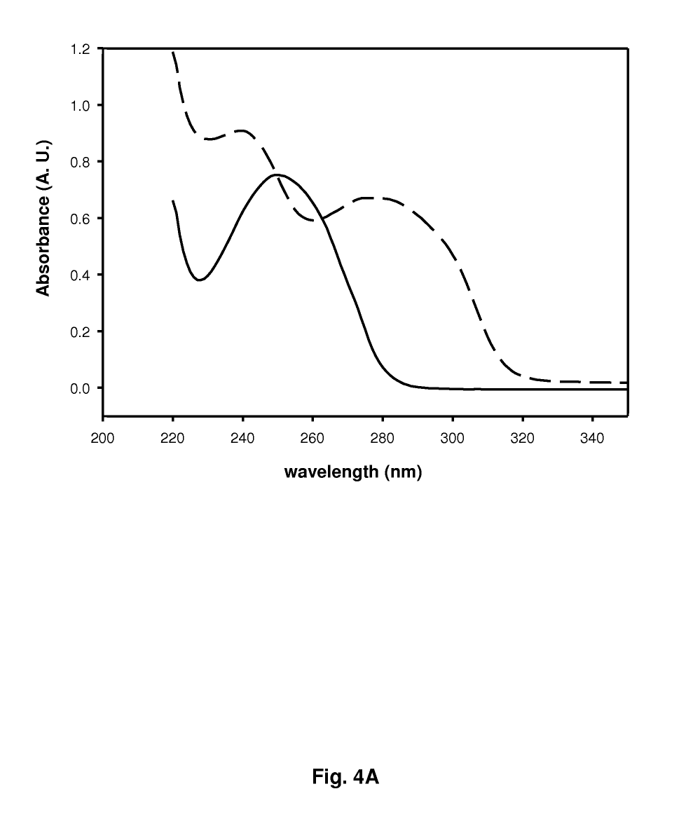

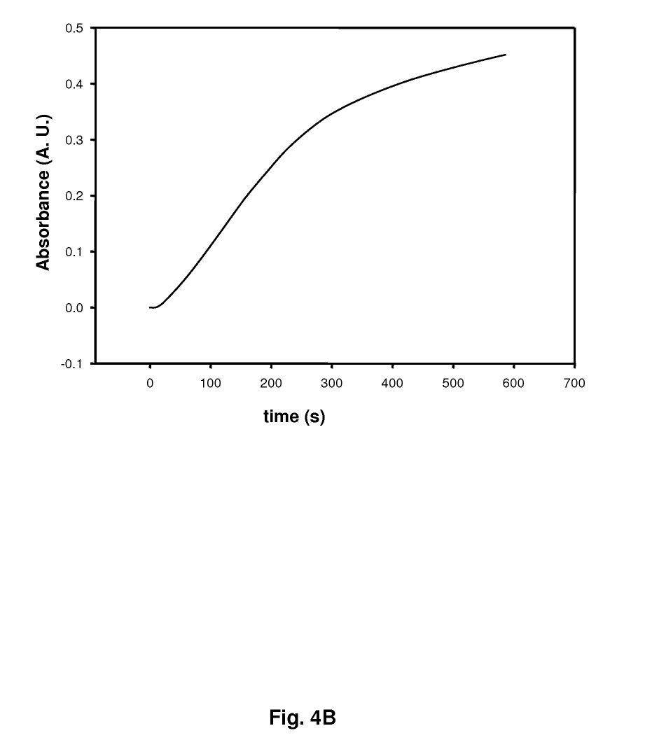

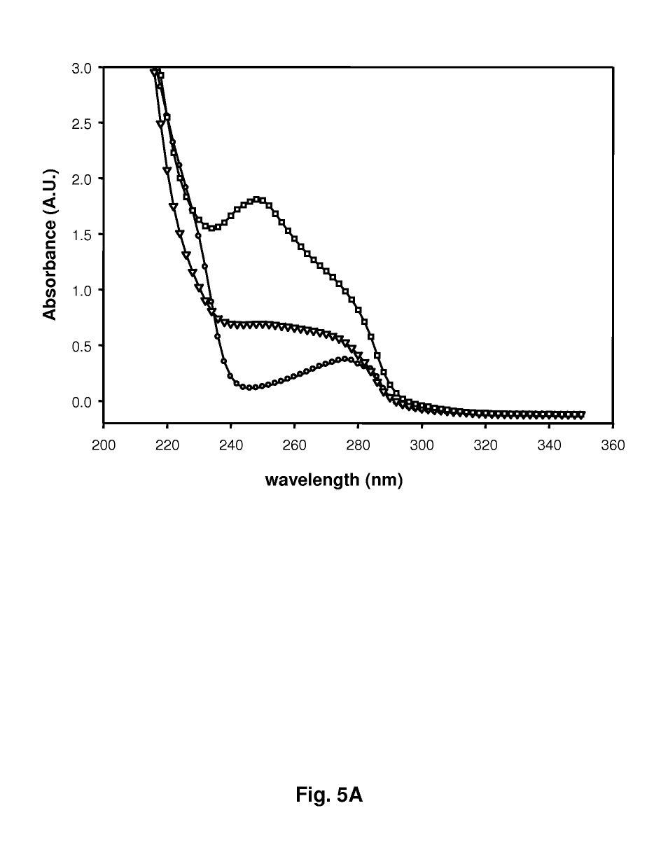

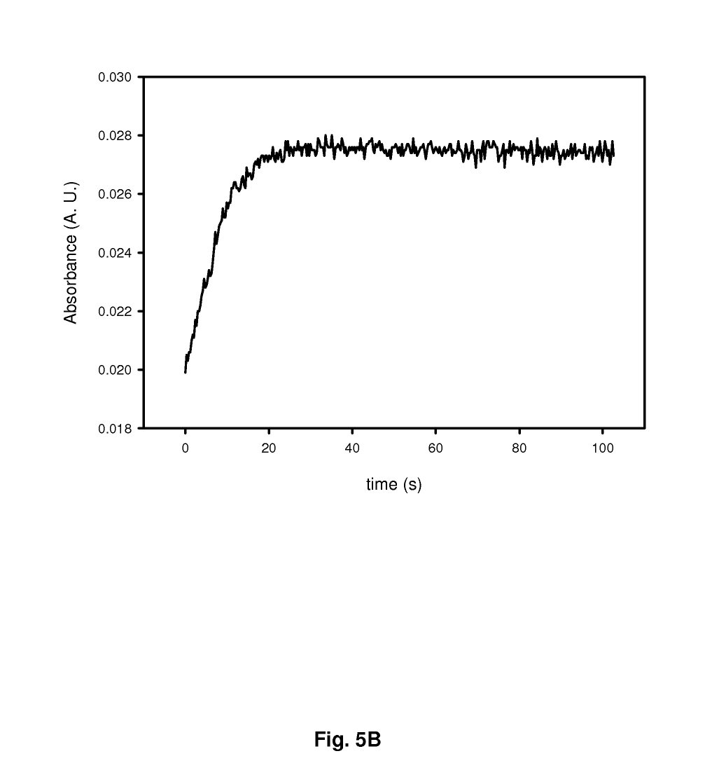

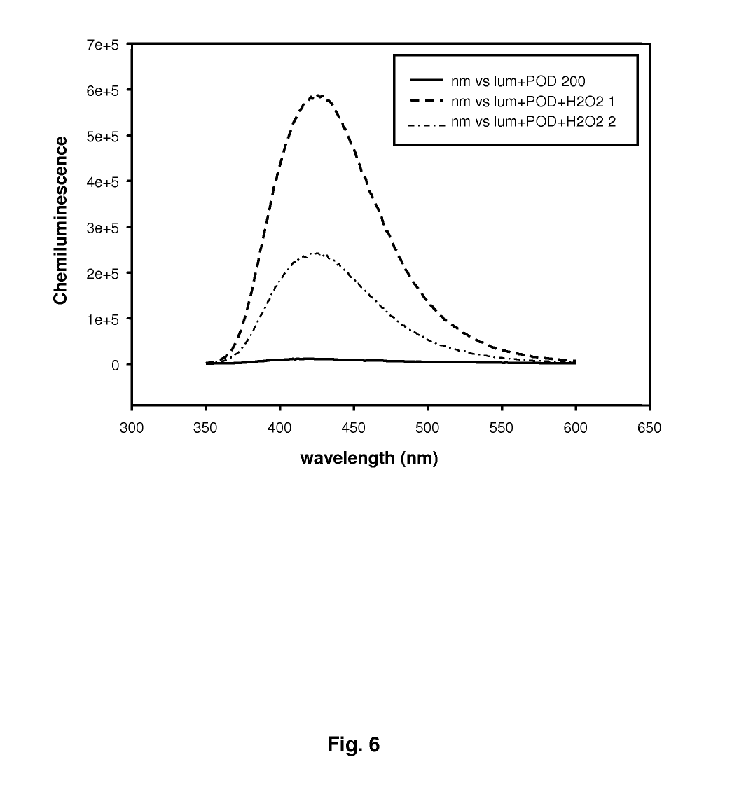

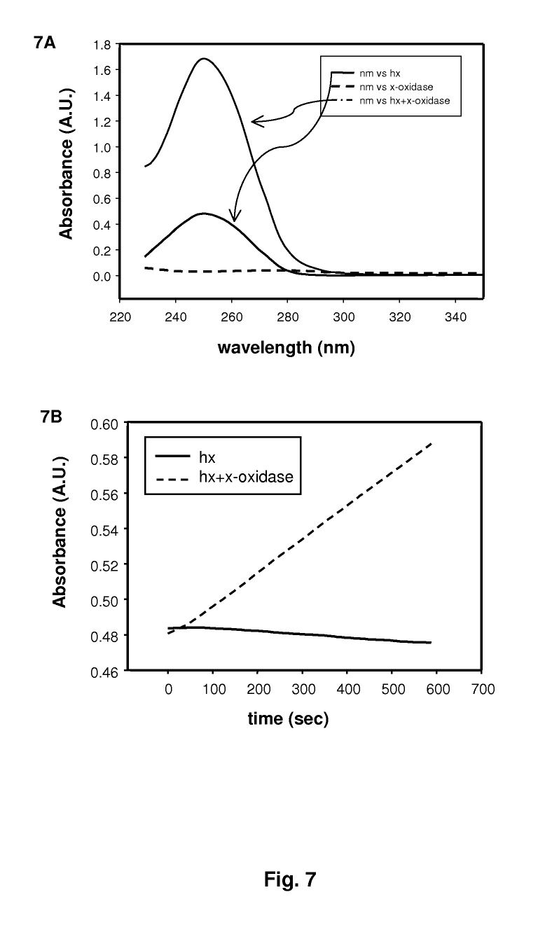

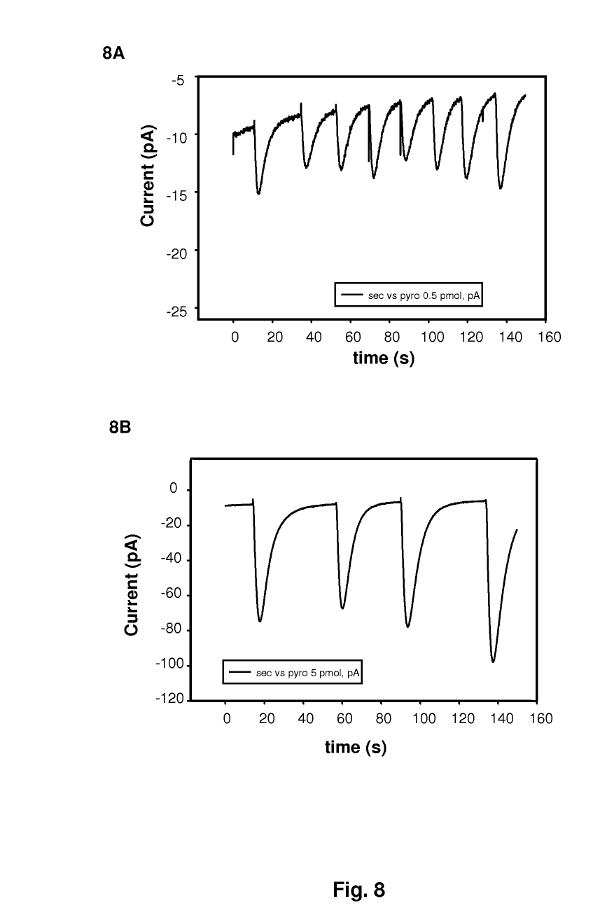

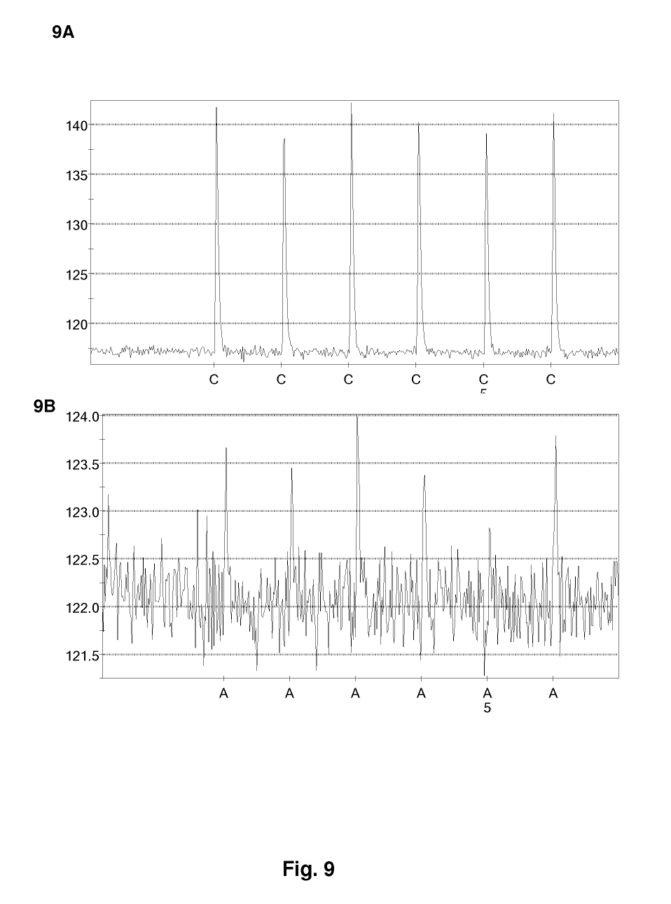

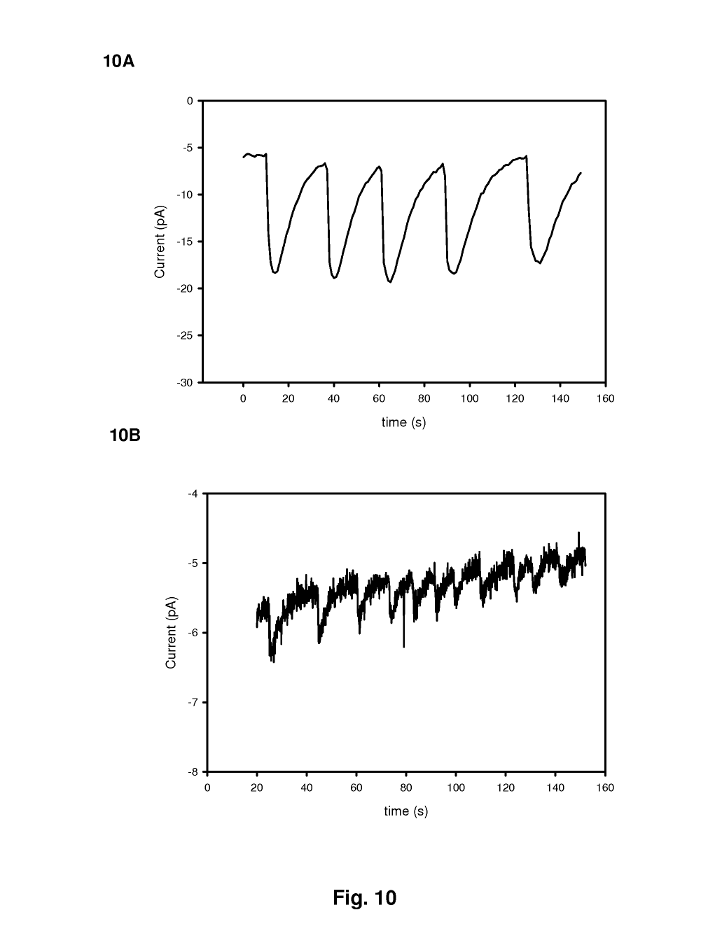

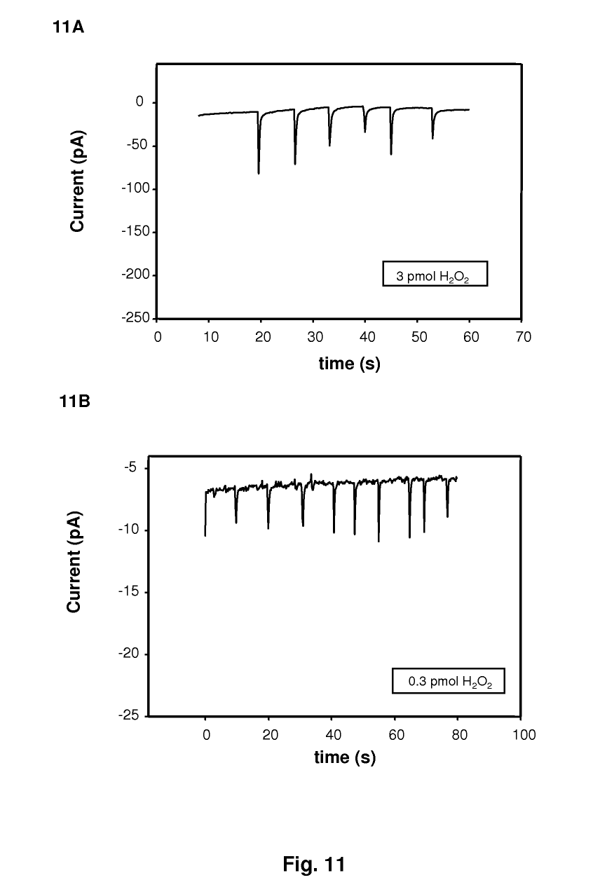

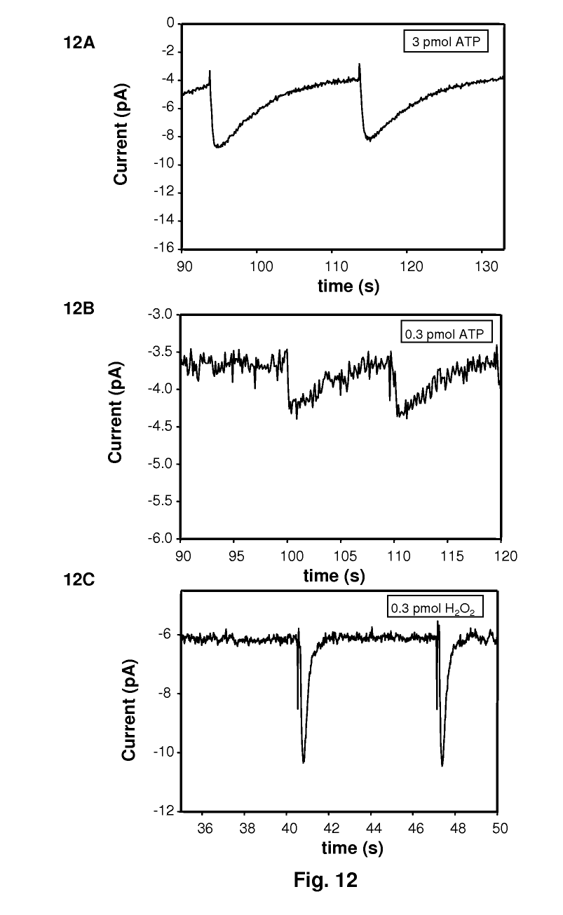

To setup the cascade of reactions, each reaction was tested independently and then combined, one reaction at a time, starting from the last reaction in the cascade (FIG. 2). Testing of the reactions to determine that they may be combined in a cascade resulted in data such as shown in FIGS. 4 through 13, inclusive. Collectively, the results shown there describe and enable the combination of reactions set forth here, and demonstrate that the reaction kinetics are suitable for a chemiluminescent oxidation assay that produces a detectable light or redox signal. The method demonstrated here involves a procedure in which one nucleotide is added at a time, in sequence. It is also contemplated that multiple, different nucleotides can be added, and the light peaks be analyzed according to their shape to show either single or multiple base incorporation.

The oxidation of luminol by POD is the last reaction in the cascade. This reaction is very fast and sensitive to hydrogen peroxide that was detected as less as 0.3 pmol (88 nM) of hydrogen peroxide (FIG. 3). The second and third reactions are catalyzed by XOD, which is a multisubunit enzyme with complex structure and chemistry. XOD is not stable as POD, and has been freshly made before each experiment to increase the sensitivity of the assay. The sensitivity of combined XOD and POD catalyzed reactions to hypoxanthine is 2-4 pmol. The decrease in the sensitivity is partially due to the absence of optimal conditions for XOD and absence of rapid mixing that decreases diffusion and reaction rates. The addition of uricase catalyzing the oxidation of urate (the fourth reaction) increases the sensitivity to hypoxanthine 1.5-2 times as expected from the stoichiometric additional increase in the production of hydrogen peroxide. The assembly of complete cascade of reactions by adding HPRT that catalyzes hypoxanthine production from IMP in the presence of pyrophosphate (the first reaction). This reaction is the most important reaction in the detection cascade because it works as the sensor of pyrophosphate. Usually, the reaction catalyzed by HPRT is the conversion of hypoxanthine to IMP, called the forward reaction. In the pyrophosphate detection assay, we use the reverse reaction, which is less studied and documented in the literature.

FIGS. 4A and B are (A) plots of an XOD activity assay, absorbance of HX (solid line) and product uric acid (dashed line), and (B) kinetics of XOD measured at 290 nm. In a similar way (not shown), a graph was generated showing RP-HPLC analysis of reactants of HPRT reactions. Shown were HPRT forward reaction reactants, which generated different peaks: a substrate PRPP, a substrate hypoxanthine, a product IMP, a side product inosine. HPRT reverse reaction reactants were also demonstrated: a product hypoxanthine, a substrate IMP, and a side product inosine. In similar data, not shown, a graph was generated showing a series of peaks from RP-HPLC. Appropriate peaks were determined in an HPLC analysis of luminol oxidation for luminol and reactants of luminol oxidation reaction catalyzed by HRP: oxidized luminol (three peaks, fig. not shown) and HPD (another peak).

### Measurement Apparatus Setup and Procedure

Various devices were used in experiments to detect chemiluminescent light. In one arrangement, we measured photodiode current by using a low-cost Chem-Clamp amplifier (Dagan Corporation, Minneapolis, Minn.) in voltage-clamp mode with signal filtering between 300 Hz-10 kHz bandwidth. The signal was further digitized by a MiniDigi digitizer (Molecular Devices) with sampling frequency at 1 kHz. The data were recorded using Axoscope software (Molecular Devices), and the same software was used for basic signal analysis. In other experiments we used also direct Powermeter 841-PE (Newport corporation). For dispensation of reagents to reaction chamber a Micro Injector-Spritzer (BioScience Tools, CA) was used. Comparison measurements were also performed on regular Pyrosequencing machine PSQ-96 (Pyrosequencing AB).

FIG. 2A outlines the reactions discussed above where luminol is reacted with hydrogen peroxide to produce light. FIG. 2B outlines an alternative reaction where the hydrogen peroxide from the fourth step is broken down to oxygen and 2H+. This reaction may be balanced in a number of ways. It may be used in an electrochemical reaction. One embodiment involves the oxidation of iron, which would take place in an acidic environment.

It may be represented as:

H₂O₂+2Fe²⁺+2H⁺->2H₂O+2Fe³⁺

Iron may also be oxidized by the hydrogen peroxide from the Fe⁺³ to the Fe⁺⁴ state. Catalase may be added to increase the rate of the oxidation. The increased oxidative state of the iron is sensed through an iron portion of an electrode in the reaction mixture. The iron at the lower oxidative state is consumed and is replenished as needed.

The change in the redox state of an iron-containing electrode may be measured by potentiometry. The redox electrode is an electrode made from electron-conductive material and characterized by high chemical stability in the solution under test (Pt, Au). It is used for measuring the redox potential of a specific redox system in solution. The correlation of an electrode potential and redox system composition (e.g., an Fe³⁺/Fe²⁺ system) can be described by the Nernst equation. In one embodiment, the redox potential difference is measured with respect to a standard electrode. A secondary standard reference electrode may be used, such as a calomel electrode or an Ag/AgCl electrode. In addition, hydrogen peroxide may be used to oxidize sodium thiosulfate to sulfuric acid. Starting from an alkaline solution, the resulting pH change can be followed using a sensitive pH detector.

Additional guidance for hydrogen peroxide sensing may be found in the field of glucose biosensors which utilize a reaction oxidizing glucose to produce gluconolactone and hydrogen peroxide. Certain glucose biosensors detect hydrogen peroxide produced by glucose oxidase. For example, U.S. Pat. No. 4,340,448 to Schiller et al. entitled “Potentiometric detection of hydrogen peroxide and apparatus therefor,” issued Jul. 20, 1982, describes an electrolytic cell which contains an electrolyte solution. A reference electrode and a working electrode are positioned within the cell and are connected to electrometer by electrical leads. The enzymes glucose oxidase with or without catalase is immobilized on support working electrode. A glucose containing substance is introduced into the electrolyte and interacts with the enzyme or enzymes to convert the glucose into gluconic acid and hydrogen peroxide. The catalase if present serves to convert most of the hydrogen peroxide into oxygen and water. The remaining hydrogen peroxide interacts with the support working electrode to generate an electrical potential. This potential is a function of the glucose concentration and is proportional to the logarithm of the glucose concentration. In the present method and device, of course, there is no need to sense glucose; the hydrogen peroxide is generated through the above described reactions.

In addition, methods may be employed as described in US 2009/0321257, entitled “Biosensor, Method of Producing the Same and Detection System Comprising the Same,” and U.S. Pat. No. 5,320,725 to Gregg, et al., issued Jun. 14, 1994, entitled “Electrode and method for the detection of hydrogen peroxide.” As disclosed in the above-mentioned Gregg et al., an electrochemical assay for H₂O₂ may involve electrooxidation of H₂O₂, usually near +0.7V (SCE), to O₂ or electroreduction, near 0.0V (SCE), to H₂O (Hall, Biosensors, Prentice Hall, Englewood Cliffs, N.J., 1991, p. 16, 135, 221, 224, 283-4; Cass, Biosensors: A Practical Approach, Oxford Univ. Press, 1990, pp. 33, 34). In the method of this patent, which may be adapted here, the electrode is used to directly detect H₂O₂ in a test sample. In this method, electrons generated at the electrode are relayed to the peroxidase enzyme through the redox polymer (e.g., epoxy) network to which the peroxidase is chemically bound.

Also, as disclosed in U.S. Pat. No. 5,518,591, issued May 21, 1996, entitled “Use of electrode system for measuring hydrogen peroxide concentration,” a change in the hydrogen peroxide concentration influences a variety of factors in the solution. Such factors are, among other things, the idle potential of the measurement electrode, the current densities measured on the polarization curve, and the zero point of the polarization curve, pH, the conductivity of the solution, and temperature. The difference of potential between the measurement electrode and the electrolytic solution, when measured in relation to the reference electrode, i.e., the idle potential of the measurement electrode, changes as a function of the hydrogen peroxide content in the solution. The direction and intensity of the change is dependent on the electrode material used. When an inert material, such as platinum, is used for the measurement electrode, the measurement mentioned above yields a so-called redox potential. The redox potential is a potential difference characteristic of the solution, and caused by redox reactions on the surface of the electrode, thereby measuring the oxidation capacity of the solution. The redox potential behaves when compared with the idle potential of an electrode made of a less noble material, less actively because the dissolving metal ion is non-existent. One may employ, in the design of this patent, an electrode selected from the group consisting of titanium, zirconium, tantalum and niobium. Preferably, measurement means such as a potentiometer is coupled to the measurement electrode, the reference electrode and the counter electrode, and the electrochemical potential of the measurement electrode which correlates to the concentration of hydrogen peroxide in the solution is measured by means of the potentiometer. Platinum electrodes have also been used to measure hydrogen peroxide. However, redox potential of such a platinum electrode behaves less powerfully, i.e., has a lower slope, when compared with the idle potential of an electrode made from a less noble material because the dissolving metal ion is non-existent. The potential difference measurement is a standard voltage measurement which can be used after the filtering and reinforcement directly as control data.

A preferred circuit arrangement is disclosed in US 2006/0105373 by Pourmand et al., published May 18, 2006, entitled “Charge perturbation detection system for DNA and other molecules.” In this embodiment, using electrochemical detection, local perturbations of charge in the solution near the electrode surface induces a charge in a polarizable gold electrode held at a set voltage. This event is detected as a transient current by a voltage clamp amplifier. Detection of single nucleotides in a sequence can be determined by dispensing individual dNTPs to the electrode solution and detecting the charge perturbations. The polymerization process generates local perturbations of charge in the solution near the electrode surface and induces a charge in a polarizable gold electrode. This event is detected as a transient current by a voltage clamp amplifier. Detection of single nucleotides in a sequence can be determined by dispensing individual dNTPs to the electrode solution and detecting the charge perturbations. The charge perturbation is induced by the generation of the hydrogen peroxide and its action upon a suitable electrode. It should be noted that an H+ ion is added to a solution in which a nucleotide is incorporated into a growing strand, as in the reaction shown in FIG. 1.

Certain alternative embodiments may be created given the teachings presented here. The present methods may be adapted for RNA sequencing. The oxidation of luminol by hydrogen peroxide may be done in the presence of a metal catalyst (e.g., sodium ferrate) at high pH to produce an excited state aminophthalate ion which emits blue light. Chloramine derivatives of amino acids also may be used to induce chemiluminescence of a luminol solution. Other oxidases may be used to generate hydrogen peroxide, although the oxidase must act upon the resultant molecule from the preceding phosphotransferase. The phosphotransferase chosen must not be ATP dependent. For example, Pollack et al., “PPi-dependent phosphofructotransferase (phosphofructokinase) activity in the mollicutes (mycoplasma) Acholeplasma laidlawi,” J Bacteriol. 1986 January; 165(1): 53-60 disclose A PPi-dependent phosphofructotransferase (PPi-fructose 6-phosphate 1-phosphotransferase, EC 2.7.1.90) which catalyzes the conversion of fructose 6 phosphate (F-6-P) to fructose 1,6-bisphosphate (F-1,6-P2) was isolated from a cytoplasmic fraction of Acholeplasma laidlawii B-PG9 and partially purified (430-fold). PPi was required as the phosphate donor. ATP, dATP, CTP, dCTP, GTP, dGTP, UTP, dUTP, ITP, TTP, ADP, or Pi could not substitute for PPi.

The specific enzymes will be selected and mixed in appropriate concentrations based on the kinetics of each enzyme in relation to the preceding and/or following step. These enzymatic steps can be modeled for optimization, according to the methods disclosed here. A method for modeling is given e.g., in Reeves et al., “Biological Systems from an Engineer's Point of View,” PLoS Biol 7(1): e1000021 doi:10.1371/journal.pbio.1000021. Each nucleotide added in a sequencing reaction will be allowed to react by incorporation into the growing chain (if the nucleotide is complementary). The PPi generated will be entered into the cascade of reactions described here, and a burst of light will be generated. Upon some decay in that light, another nucleotide will be added for detection of the next base to be incorporated. This overall process is as described in connection with pyrosequencing (See, U.S. Pat. No. 6,828,100 to Ronaghi, issued Dec. 7, 2004, entitled “Method of DNA sequencing,” for full details of the process flow which produces sequence information of a polynucleic acid (DNA) of unknown sequence.)

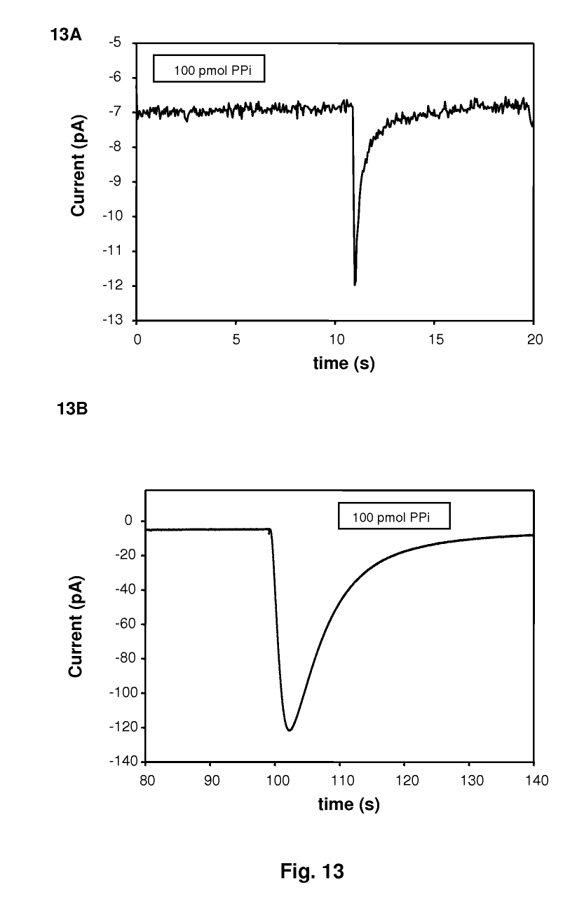

The sequence information is contained in peaks of light output. Each addition to the growing chain (polymerization) causes a peak. Unsynchronized polymerization takes place because there are typically multiple copies of the strand being sequenced that are present in the reaction mixture (e.g., on a bead). If, say an “A” is added and happens to match a “T”, the A will be incorporated into the growing strand. This has to happen in all strands in the mixture to maintain the sharp peaks, as illustrated in FIGS. 4, 5, 6, 7A, 10, 11, 12 and 13. FIG. 13 shows the overall light output from the combined reactions in comparing pyrosequencing (A) and peroxysequencing (B). The sharpness of the peak in time is important when numerous base reads are to be carried out.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to. The specific aspects of the incorporated document to be incorporated will be apparent from the context.

What is claimed is:

1. A method of detecting incorporation of a nucleotide into a polynucleotide, whereby PPi is generated, comprising the steps of:
(a) combining said PPi with a phosphotransferase to form an oxidatable compound;
(b) in a second step, oxidizing the oxidatable compound to form hydrogen peroxide;
(c) then contacting the hydrogen peroxide with a light emitting compound; and
(d) detecting a signal, caused by reaction of the hydrogen peroxide with the light emitting compound, as indicative of incorporation of the nucleotide.

2. The method of claim 1, using enzymes hypoxanthine-phosphoribosyl transferase in step a, xanthine oxidase in step b, and peroxidase in step c.

3. The method of claim 1 where the polynucleotide is DNA.

4. The method of claim 1 where the enzymes hypoxanthine-phosphoribosyl transferase in step a, xanthine oxidase in step b, and peroxidase in step c are added sequentially.

5. The method of claim 4 where the enzymes are simultaneously present in the reaction mixture when a PPi is generated.

6. The method of claim 1 where the light emitting compound is a luminol compound.

7. The method of claim 1 where the oxidatable compound is hypoxanthine.

8. The method of claim 7 where the oxidatable compound is oxidized with the use of xanthine oxidase.

9. The method of claim 8 where the contacting of hydrogen peroxide is accompanied by reacting with peroxidase.

10. The method of claim 9 where the oxidatable compound is luminol.

11. The method of claim 10 where the phosphotransferase is HPRT.

12. The method of claims 11 further comprising the step of adding a uricase enzyme for further converting a urate product derived from step (b) to produce hydrogen peroxide.

13. A method of detecting the incorporation of a nucleotide into a polynucleotide, whereby PPi is generated, comprising the steps of:
(a) combining said PPi with a phosphotransferase to form an oxidatable compound;
(b) in a second step, oxidizing the oxidatable compound to form hydrogen peroxide;
(c) then decomposing the hydrogen peroxide to form oxygen and protons; and
(d) detecting a signal in an electrode generated as a result of the generation of said protons, as indicative of incorporation of the nucleotide.

14. The method of claim 13 wherein decomposing of the hydrogen peroxide comprises oxidation of metal in an electrode.

15. The method of claim 13 wherein decomposing of the hydrogen peroxide generates a charge differential sensed by an electrode held at a set voltage.

16. A kit for sequencing DNA, comprising HPRT.

17. The kit of claim 16 further comprising xanthine oxidase.

18. The kit of claim 16 further comprising peroxidase.

19. The kit of claims 16 further comprising apyrase.

20. The kit of claims 16 further comprising DNA polymerase.

21. The kit of claims 16 further comprising RNA polymerase.

22. The kit of claims 16 further comprising uricase.
